(19) United States
(12) Patent Application Publication
(10) Pub. No.: US 2017/0258398 A1
(43) Pub. Date: Sep. 14, 2017

(54) CHILD MONITORING SYSTEM

(71) Applicant: KIDS II, INC., Atlanta, GA (US)

(72) Inventor: Peter JACKSON, Alpharetta, GA (US)

(73) Assignee: KIDS II, INC., Atlanta, GA (US)

(21) Appl. No.: 15/456,888

(22) Filed: Mar. 13, 2017

Related U.S. Application Data
(60) Provisional application No. 62/306,743, filed on Mar. 11, 2016.
(60) Provisional application No. 62/394,402, filed on Sep. 14, 2016.
(60) Provisional application No. 62/394,417, filed on Sep. 14, 2016.

(51) Int. Cl. A61B 5/00; A61M 21/02; A61B 5/05; A61B 7/04; A61B 5/01

(52) U.S. Cl. CPC A61B 5/4812; A61B 5/0077; A61B 7/04; A61B 5/01; A61B 5/0507; A61B 5/742; A61B 5/7405; A61M 21/02; A61M 2021/0027

(57) ABSTRACT

A system for collecting and/or communicating information to a caregiver or other user relating to the sleep and/or development of a child. Example embodiments of the invention are configured to collect and/or relay information relating to the sleep of a child and automatically implement a sequence of child-soothing measures to calm down a crying infant before resorting to alerting the caregiver. For example, the system can be configured to implement soothing measures such as white noise, vibration, soft music, rocking, etc. Other example embodiments of the invention are additionally or alternatively configured to collect and/or relay information relating to the cognitive and/or physical development of a child. For example, the system can be configured to measure various indicators related to a child's development and/or to provide feedback to a caregiver regarding the measured data.

### CROSS-REFERENCE TO RELATED APPLICATIONS

This application claims the benefit of U.S. Provisional Patent Application Ser. No. 62/306,743 filed Mar. 11, 2016, U.S. Provisional Patent Application Ser. No. 62/394,402 filed Sep. 14, 2016 and U.S. Provisional Patent Application Ser. No. 62/394,417 filed Sep. 14, 2016, the entireties of which are hereby incorporated herein by reference for all purposes.

### TECHNICAL FIELD

The present invention relates generally to the field of childcare products, and more particularly to a system and method for monitoring and/or improving children's sleep and/or development.

### BACKGROUND

Infants tend to have difficulty sleeping for extended periods of time, often awaking in the middle of the night in a fit of crying and screaming. Conventional teaching is for caregivers to allow the child to cry for incrementally longer periods of time before going to the child and comforting and calming it down. But this is very distressful not only for the child but also for the parents or other caregiver.

In addition, it is known that approximately 80 percent of a child's cognitive development takes place during the child's first five years of life. Products on the market that foster or claim to foster a child's cognitive or physical development are well-known in the market. However, these products do not provide feedback on the child's cognitive or physical progress to the child's caregiver. Thus, a caregiver does not know whether the product is aiding their child's development, or whether their child is developing at a desired rate.

Accordingly, it can be seen that needs exist for improvements in child sleep-care and/or cognitive development. It is to the provision of solutions to these and other needs that the present invention is primarily directed.

Summary

Generally, the present invention relates to a system for collecting and/or communicating information to a caregiver or other user relating to the sleep and/or development of a child. Example embodiments of the invention are configured to collect and/or relay information relating to the sleep of a child and automatically implement a sequence of child-soothing measures to calm down a crying infant before resorting to alerting the caregiver. For example, the system can be configured to implement soothing measures such as white noise, vibration, soft music, rocking, etc. Other example embodiments of the invention are additionally or alternatively configured to collect and/or relay information relating to the cognitive and/or physical development of a child. For example, the system can be configured to measure various indicators related to a child's development and/or to provide feedback to a caregiver regarding the measured data.

According to one aspect of the invention, the system comprises a monitoring device for measuring indicators related to the sleep and/or development of a child. According to another aspect of the invention, the system comprises a feedback device for outputting measured data related to a child's sleep and/or development. According to another aspect of the invention, the system comprises a monitoring device and a feedback device. The monitoring device measures indicators related to the sleep and/or development of a child and relays the measured data to the feedback device. The feedback device can output the data to a caregiver or other user. The feedback device can additionally or alternatively output alerts to the caregiver relating to the monitored child's sleep condition, and the monitoring device can additionally or alternatively implement child-soothing measures before sending the alerts.

These and other aspects, features, and advantages of the invention will be understood with reference to the drawing figures and detailed description herein, and will be realized by means of the various elements and combinations particularly pointed out in the appended claims. It is to be understood that both the foregoing summary description and the following brief description of the drawings and detailed description of example embodiments are explanatory of example embodiments of the invention, and are not restrictive of the invention, as claimed.

### BRIEF DESCRIPTION OF THE DRAWINGS

FIG. 1 is a system block diagram of a monitoring/feedback system according to a first example embodiment.

FIG. 2 is a system block diagram of a monitoring/feedback system according to a second example embodiment.

FIG. 3 is a perspective view of a feedback device according to a third example embodiment.

FIG. 4 is a perspective view of children's sleep unit incorporating a sleep monitoring/feedback system according to a fourth example embodiment.

FIG. 5 is a perspective view of children's toy incorporating a learning monitoring device according to a fifth example embodiment.

FIG. 6 is a perspective view of children's swing incorporating a swing monitoring device according to a sixth example embodiment.

FIG. 7 is a perspective view of children's entertainer incorporating an exercise monitoring device according to a seventh example embodiment.

FIG. 8 is a system block diagram of a monitoring/feedback system according to an eighth example embodiment.

FIG. 9 is a plot of a sleep cycle showing REM sleep entered before stage 1 sleep.

FIG. 10 is a perspective view of children's sleep unit incorporating a sleep monitoring/feedback system according to a ninth example embodiment.

FIG. 11 is a system block diagram of a monitoring/feedback system according to a tenth example embodiment.

FIG. 12 is a process flow diagram for example operational control of the system of FIG. 13 to implement a sleep-training program.

FIG. 13 is a side view of the monitoring/feedback system of FIG. 11 mounted to a children's sleep unit for use.

FIG. 14 shows the monitoring/feedback system of FIG. 13 detached from its sleep-unit mount.

FIG. 15 is a screen display of of a user interface showing outputs and inputs from and to the control unit of the monitoring/feedback system of FIG. 11.

### DETAILED DESCRIPTION OF EXAMPLE EMBODIMENTS

The present invention may be understood more readily by reference to the following detailed description of example embodiments taken in connection with the accompanying drawing figures, which form a part of this disclosure. It is to be understood that this invention is not limited to the specific devices, methods, conditions, or parameters described and/or shown herein, and that the terminology used herein is for the purpose of describing particular embodiments by way of example only and is not intended to be limiting of the claimed invention. Any and all patents and other publications identified in this specification are incorporated by reference as though fully set forth herein.

Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

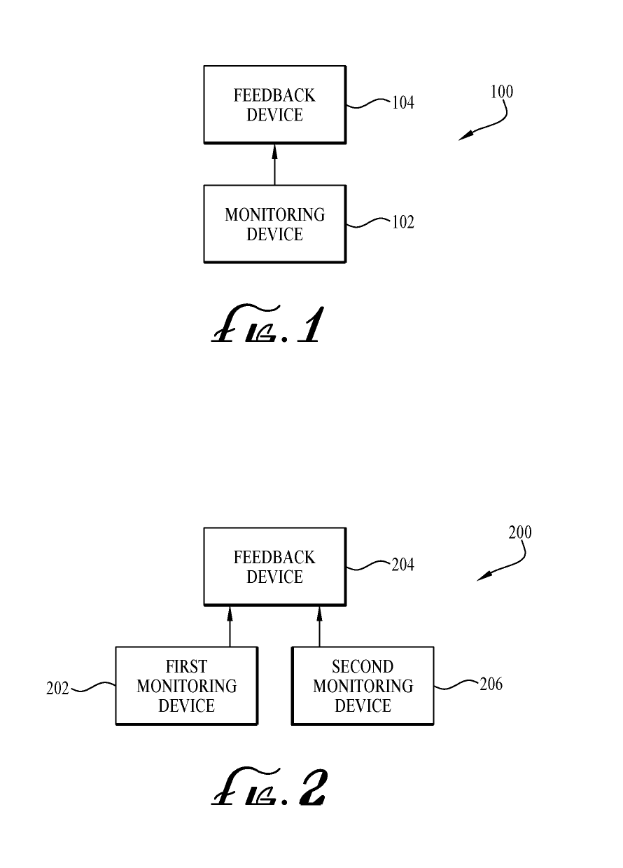

With reference now to the drawing figures, wherein like reference numbers represent corresponding parts throughout the several views, FIG. 1 shows a child-development monitoring and feedback system 100 according to a first example embodiment of the present invention. The monitoring/feedback system 100 includes a monitoring device 102 and a feedback device 104 in communication with the monitoring device for receiving feedback from it.

The monitoring device 102 can be configured to monitor one or several influencers or indicators of cognitive development, such as sleep, stimulus (play), nutrition, and/or stress. For example, in the depicted embodiment, the monitoring device 102 is a sleep-monitoring device, as sleep plays a large role in the cognitive development of children. The sleep monitoring device 102 can measure, for example, the sleep quality, quantity, and patterns of a child. It can also be configured to monitor a child's sleep cycles and to recognize what stage of sleep a child is experiencing. To provide this input functionality, the monitoring device can include one or a plurality of input devices/sensors 106 positioned proximate the child (close enough to detect/receive the inputs, including near the child, on the child's clothing, strapped to the child, etc.) and configured for measuring various indicators related to sleep, such as movement, breathing rate, heart rate, body temperature, brain activity, eye movement, etc. The input devices can thus include, for example, a motion sensor, a camera with computer vision, a low-power radar, electrodes, a thermometer, a microphone, etc., of a conventional type well-known in the art. In typical embodiments, the monitoring device 102 also includes one or more control devices (e.g., an on-off button, input selector switch(es), unit selection settings such as temperature in degrees C. or F) and a conventional processor programmed for implementing the desired control functionality.

The monitoring device 102 is configured to send the measured data (e.g., number of hours slept in a night, number of sleep cycles completed) to the feedback device 104. In typical embodiments, the monitoring device 102 includes a conventional transmitter and antenna and the feedback device 104 includes a conventional antenna and receiver for conventional wireless communications from the monitoring device to the feedback device. In other embodiments, the monitoring device communicates with the feedback device by communications wiring or other conductors electrically connected between the two components. In typical embodiments, the monitoring device 102 and the feedback device 104 are two separate components that can be remotely positioned relative to each other so that the child can be sleeping or playing in one room of a house and the caretaker can be in another room in the same house. In other embodiments, the monitoring device and the feedback device are provided as a one-piece unit, and they are integral (they cannot be detached from each other) or they are detachable (they can be separated and remotely positioned relative to each other). In two-piece embodiments each component includes a power source (e.g., battery and/or power cord) or is in electrical-power communication (e.g., power wiring or other conductors) with a power source of the other components, and in one-piece embodiments a single power source can run both components.

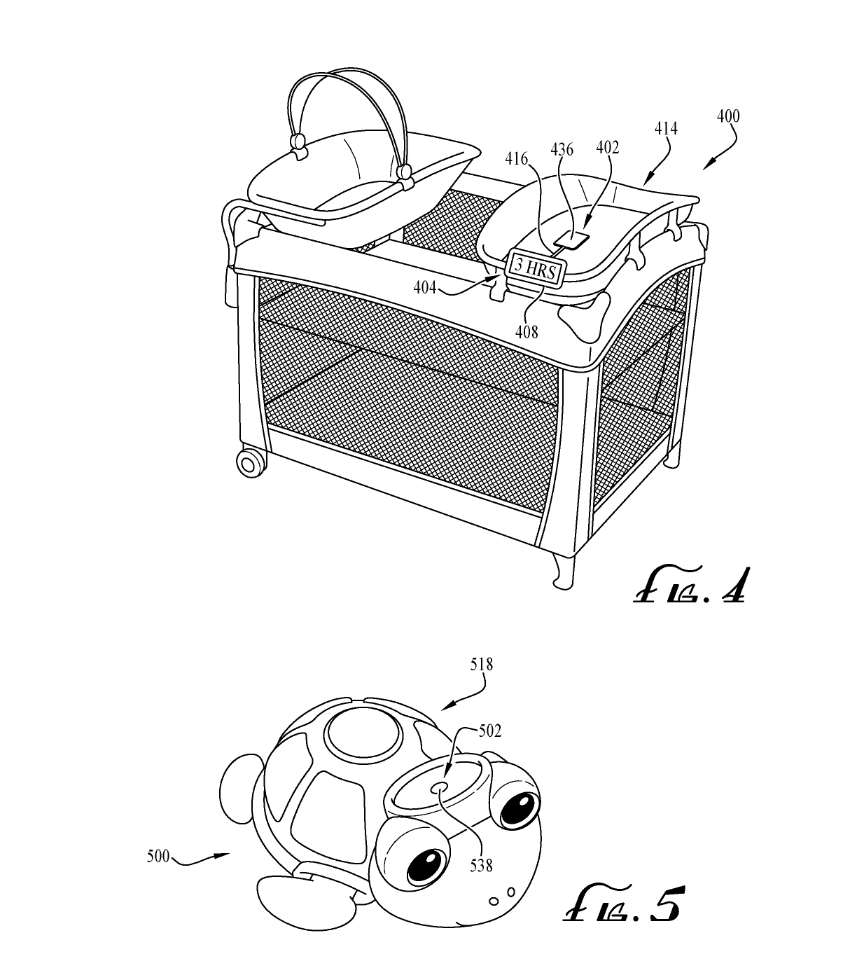

The feedback device 104 receives the measured data from the monitoring device 102 and outputs the data to a caregiver in a user-friendly form. For example, the feedback device 104 can audibly announce and/or visually display the data (e.g., “Your child slept 6 hours last night”). As such, the feedback device 104 includes at least one output device such as a speaker, display screen, light, and/or vibrator of a conventional type well-known in the art. In typical some embodiments, the feedback device 104 also includes one or more control devices (e.g., an on-off button, a volume and/or brightness control) and a conventional processor programmed for implementing the desired control functionality. Further, the feedback device 104 can be physically integrated into a conventional children's product (e.g., as shown and described with respect to FIG. 3), provided as a software product (e.g., an Internet website ora mobile-device application) for loading onto and using communication and output components of a computer device (e.g., a desktop or laptop, or a smart phone or tablet), or implemented using another conventional platform through which information can be communicated and output.

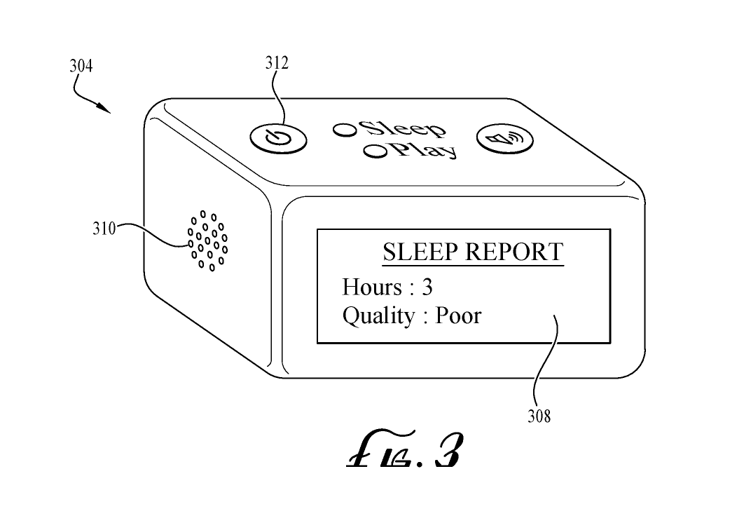

FIG. 2 shows a child-development monitoring and feedback system 200 according to a second example embodiment of the present invention. The monitoring/feedback system 200 includes a first monitoring device 202, a second monitoring device 206, and a feedback device 204 in communication with the first and second monitoring devices for receiving feedback from them. The first monitoring device 202 can be, for example, a sleep monitoring device, as described above, and the second monitoring device 206 can be, for example, a learning monitoring device, as described below. The sleep monitoring device 202 and the learning monitoring device 206 are configured to send measured data to the feedback device 204. The feedback device 204 receives the data from the first and second monitoring devices 202 and 206 and outputs the data to a caregiver.

The second/learning monitoring device 206 includes input devices/sensors configured to measure indicators of a child's cognitive learning. For example, the learning monitoring device 206 can be embedded in a toy whose use by a child encourages number learning such as by measuring the accuracy with which a child recognizes numbers. In one example embodiment, the learning monitoring device 206 includes an eye-tracking input device of a conventional type well-known in the art, which tracks a child's eye movements when a command is given by an output device of the first monitoring device. For example, if the command “Find the number 3” is given by a display/visual or speaker/audible output device of the monitoring device 206, the eye-tracking input device (e.g., a camera in communication with a programmed processor) can determine if the child looks at the correct number. In another example embodiment, the learning monitoring device 206 can track a child's physical interaction with the device. In such embodiments, the learning monitoring device 206 can include tactile response input devices such a push buttons or push pads of a conventional type well-known in the art. For example, if the command “Touch the number 3” is given, the learning monitoring device 206 can determine (e.g., as a binary process) if a child touches the correct number within a preset time.

FIG. 3 shows a feedback device 304, according to a third example embodiment, that can be included in for example the embodiments of FIG. 1 or 2. The feedback device 304 can be configured to wirelessly receive information from a monitoring device. According to other embodiments, the feedback device 304 can be configured to receive information from a monitoring device via an alternative transmitter, such as a wire. The feedback device 304 can be separate from a monitoring device, as depicted, or it can be integrally formed with a monitoring device into a one-piece unit. The feedback device 304 can include an output device in the form of a display screen 308 on which is shown information measured by and received from a monitoring device, as depicted. The feedback device 304 can also include an output device for example in the form of a speaker 310 to audibly relay such information to the caregiver. Example embodiments can also include a user interface 312 for entering preset parameters and operating the output devices such as an on/off power button, a volume button, and other user selections.

FIGS. 4-6 illustrate various conventional children's products adapted to include monitoring and/or feedback devices and thus implement various example embodiments of the monitoring and/or feedback systems of FIG. 1 or 2. In some embodiments, the monitoring and/or feedback devices are physically integrated into children's products during manufacture, and in others they are provided as aftermarket retrofit kits for user attachment to the children's products.

In a fourth example embodiment of FIG. 4, the sleep monitoring device 402 is physically integrated into a conventional children's sleep unit 414. As described above, the sleep monitoring device 402 can include one or more input devices/sensors to measure, for example, the sleep quality, quantity, sleep patterns, or sleep cycles of a child. In the depicted example, the monitoring device 402 includes a motion sensor 436 for detecting the motions (e.g., occurrence and duration of each) of a sleeping child occupant. In other embodiments, a sensor can additionally or alternatively monitor (e.g., detect and measure) breathing rate, heart rate, brain activity, body temperature, eye movement, facial cues, or other indicators of sleep conditions. The input devices/sensors can thus include, for example, a motion sensor, a camera with computer vision, a low-power radar, electrodes, a thermometer, a microphone, etc., of a conventional type well-known in the art.

The sleep monitoring device 402 is configured to send the motion data to a feedback device 404 via a communications wire 416. The feedback device 404 can include a conventional processor (not shown) and a conventional storage device (not shown) adapted to process and interpret the data and to store the raw and/or processed data. And the feedback device 404 can include one or more output devices to communicate information about the child's sleep to a caregiver, for example, a display screen 408 can be provided for visually communicating to the caregiver and/or a speaker or other audio output unit (not shown) can be provided for audibly communicating to the caregiver.

The monitoring/feedback system 400 can also be adapted to include various input devices/sensors to monitor external factors that influence sleep, physical comfort, and/or wellbeing. The monitoring/feedback system 400 can further be adapted to include various output devices and corresponding control devices to alter the external factors to improve the child's comfort or sleep environment. The monitoring/feedback system 400 can further be adapted to include a conventional data-storage device that saves preset desired-factor data (e.g., minimum and/or maximum temperature and/or noise level) and a programmed processor that compares that to the measured-factor data, with these components typically included in the feedback device 404.

In one such example, the monitoring/feedback system 400 monitors the external factor of the air temperature and automatically adjusts a room/house's thermostat settings if the air temperature is outside of a preferred range. For example, the monitoring/feedback system 400 can include a monitoring device 402 with a dedicated thermometer input device (e.g., mounted to or formed into the children's sleep unit 414, not part of the home thermostat) for monitoring the temperature proximate the sleeping child, a storage device with a saved user-selected preset minimum and/or maximum temperature (e.g., in a database), a programmed processor for comparing the monitored temperature with the preset temperature to determine any variance and any action required to correct the variance, a communications system (e.g., transmitter/antenna) for sending a control signal to the room/house's wireless-controlled thermostat to turn on or off the house's HVAC system to correct the variance.

Alternatively or additionally, the monitoring/feedback system 400 can monitor the child's body temperature and automatically adjust the thermostat settings if the child is too hot or too cold. For example, the monitoring/feedback system 400 can include a monitoring device 402 with a thermometer input device (e.g., attached to or formed into a warming or cooling blanket or pad in the children's sleep unit 414, or included in a wristband worn by the child) for monitoring the temperature proximate the sleeping child, a storage device with a saved user-selected preset minimum and/or maximum temperature (e.g., in a database), a programmed processor for comparing the monitored temperature with the preset temperature to determine any variance and any action required to correct the variance, a communications system (e.g., control and/or power wiring) for sending a control or power signal to the heating or cooling element of the heating or cooling blanket, pad, or another external device with temperature control, to turn it on/off or up/down to correct the variance.

According to another example embodiment, the monitoring/feedback system 400 monitors ambient noise and automatically activates an audio unit (e.g., a white noise machine) to counteract disruptive ambient noise with soothing and/or noise-cancelling sounds. For example, the monitoring/feedback system 400 can include a monitoring device 402 with a microphone input device (e.g., mounted to or formed into the children's sleep unit 414) for monitoring the ambient noise/sound level proximate the sleeping child, a storage device with a saved user-selected preset minimum and/or maximum noise/sound level (e.g., in a database), a programmed processor for comparing the monitored noise/sound level with the preset noise/sound level to determine any variance and any action required to correct the variance, a communications system (e.g., control and/or power wiring) for sending a control or power signal to the audio unit to turn its speaker output device on/off or up/down to correct the variance.

And the monitoring/feedback system 400 can monitor an actual sleep factor and provide feedback to a caregiver on whether their child's sleep is meeting a recommended standard for that sleep factor. For example, if the recommended amount of REM sleep per day for an infant is at least five hours, the desired-factor REM-sleep data can be preset by the caregiver to five hours minimum. In an example embodiment, the monitoring/feedback system 400 can include a monitoring device 402 with a microphone input device (e.g., mounted to or formed into the children's sleep unit 414 in a location proximate the sleeping child's head) for monitoring (detecting and converting into an electrical signal) the child's sleep sounds, a storage device with the saved recommended/desired-factor REM-sleep data (e.g., five hours minimum), a programmed processor for analyzing the input sleep sounds to determine the child's actual REM-sleep data (i.e., how much REM sleep a child experiences per day) then comparing that to the recommended/desired-factor REM-sleep data to determine any variance, a communications system (e.g., transmitter/antenna) for sending a data signal to the feedback device 404, and a display screen of the feedback device 404 for communicating to the caregiver whether the child is meeting the recommended REM-sleep goal for a child that age.

The feedback device 404 can also be adapted to recommend solutions or connect a caregiver with resources for improving a child's sleep if it determines the child is not getting the recommended REM sleep. For example, the feedback device 404 can provide a link to a reputable website with information on children's sleep, with its transmitter/antenna sending an email or text to a user device (e.g., a conventional computer/communications device) by wireless signal (e.g., RF or cellular). In some embodiments, the feedback device 404 can communicate the measured data to the child's pediatrician or a sleep expert, and the pediatrician or sleep expert can provide comments or recommendations back to the caregiver through the feedback device or through other conventional communication technologies.

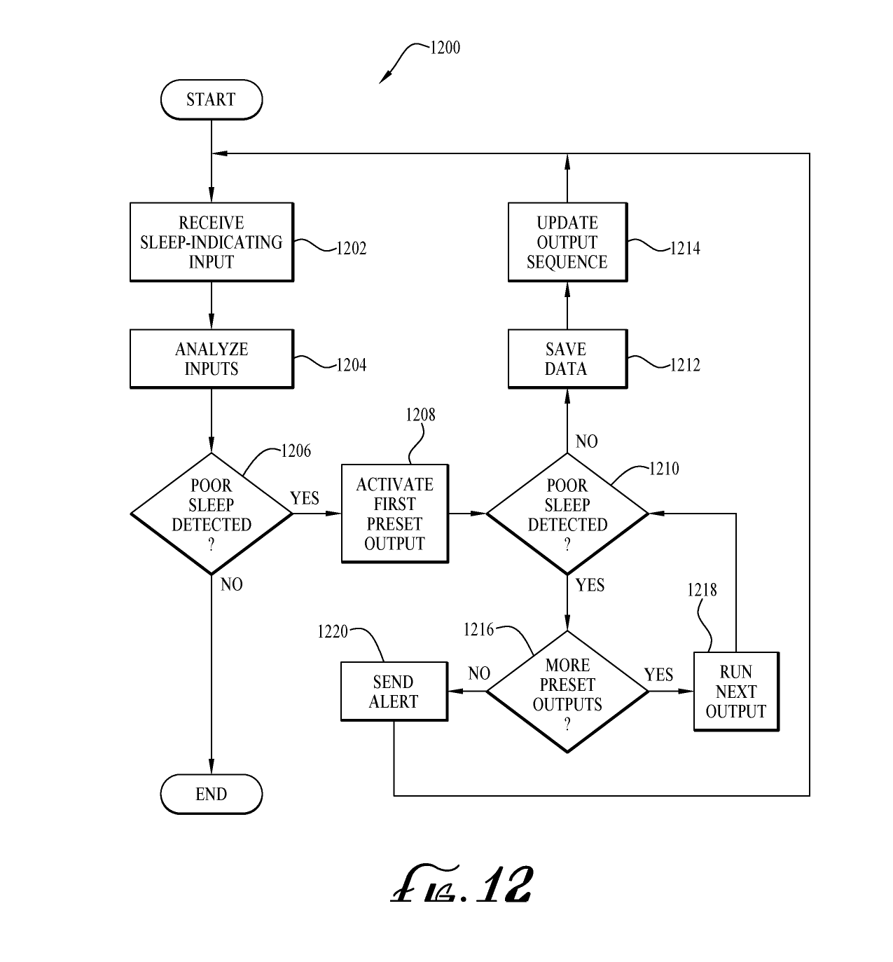

The monitoring/feedback system 400 can also be adapted to “learn” a child's preferences. In one example, the monitoring/feedback system 400 can include an audio unit including various soothing sounds. The monitoring system 400 can determine that a child sleeps better or cries less when a certain sound is playing and can automatically turn on the preferred sound when the monitoring device senses disrupted sleep or crying. Additional structural and programming details and variations of such embodiments of the monitoring/feedback system 400 are described herein with respect to FIGS. 11-15.

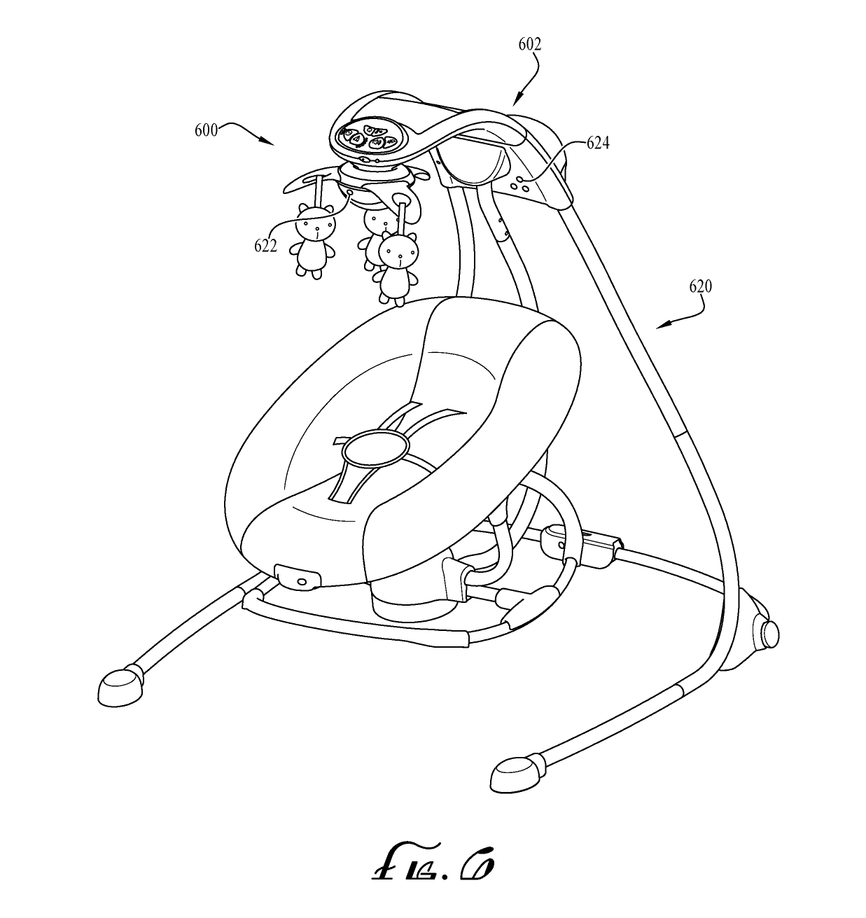

In a fifth example embodiment of FIG. 5, a monitoring/feedback system 500 is configured to measure indicators of cognitive learning. The monitoring/feedback system 500 includes a learning monitoring device 502 that is embedded in a toy 518 that encourages color recognition and is configured to measure a child's recognition of colors. For example, if the command “Touch the purple button” is given, the learning monitoring device 502 can determine if a child presses the correctly colored button. The learning monitoring device 502 can also be configured to encourage body part recognition. As shown, the monitoring device 502 can include a camera 538 that tracks a child's physical movements. If the command “Point to your tummy” is given (e.g., selected by a programmed processor from a menu of commands saved on a storage device and output audibly by a speaker of the monitoring/feedback system 500), the learning monitoring device 502 can determine (e.g., by the programmed processor analyzing/interpreting the input image signals from the camera) if the child points to the correct body part. In other examples, the monitoring device 502 can monitor a child's facial cues or eye movements as indicators of recognition, understanding, frustration, etc. (e.g., using the same input, output, storage, and processing devices. Example embodiments of the monitoring/feedback system 500 can include a feedback device, such as the remote feedback device 304 of FIG. 3, which receives learning information from the learning monitoring device 502 and includes a programmed processor for processing the information and evaluating the child's cognitive abilities and progress over time. In alternative embodiments, the feedback device can be an app downloadable to a caregiver's mobile electronic device. The monitoring system 500 can be adapted to change the content of the toy 518 based on the child's abilities and progress. For example, if a child has demonstrated understanding of the toy's current content (e.g., color recognition), the content can be changed to new or more advanced content (e.g., number recognition). Or, the monitoring system 500 can be adapted to learn a child's preferences, for example, by tracking a child smiling using computer vision. The monitoring system 500 can be adapted to customize the toy's content based on the child's preferences. Further, the feedback device can be adapted to communicate the child's progress and/or preferences to a caregiver. The feedback device can also be adapted to communicate the child's progress to a pediatrician or learning expert and to provide resources/recommendations based on the child's progress, similar to the sleep feedback device 404 described above. Additional structural and programming details and variations of such embodiments of the monitoring/feedback system 500 are described herein with respect to FIGS. 11-15.

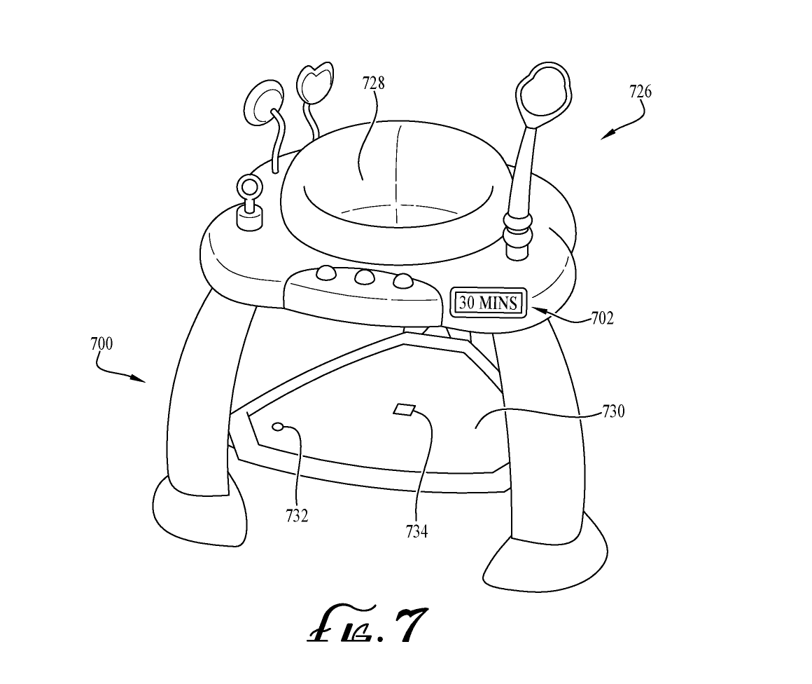

In a sixth example embodiment of FIG. 6, another monitoring/feedback system 600 is shown. The monitoring/feedback system 600 includes a swing monitoring device 602 coupled to and incorporated into a conventional child swing 620. Various aspects of the swing 620 can be adjustable, for example, the speed of the swing and/or the type of motion (e.g., front to back, side to side, etc.) by conventional swing-adjustment mechanisms well-known in the art. The swing 620 can also include other adjustable child-engagement mechanisms, such as a music unit (e.g., with an adjustable-volume speaker and/or song-menu storage and display devices and an interoperable song-selector device), a vibration unit (e.g., with adjustable intensity), etc. The swing device 620 can include an eye-tracking device (e.g., a video camera) 622 that can determine when a child's eyes are opened (i.e., the child is awake) and when they are closed (i.e., the child is asleep). When the eye-tracking device detects (e.g., by a programmed processor analyzing/interpreting its input images) 622 that the child's eyes are closed, the monitoring device 602 can identify the current settings of the swing device 620 (e.g., speed, motion type, vibration intensity, lullaby played, music volume, etc.), which represent preferred settings for promoting the child falling and/or staying asleep. The preferred settings can be relayed to a feedback device (not shown; e.g., the remote feedback device 304 of FIG. 3), which can be attached to or separate from the swing device 620. The feedback device can then communicate the preferred settings to a caregiver. In example embodiments, the preferred settings can be saved as a pre-defined “sleep setting,” which can be selected by a caregiver whenever desired.

In another example, the monitoring device 602 can include a sound detector 624 (e.g., microphone and programmed processor) which can distinguish between various sounds, for example, silence (i.e., the lack of any detected sound), happy sounds (e.g., laughter), and agitated sounds (e.g., crying). The monitoring device 602 can then determine which settings the child likes and dislikes, and relay this information to the caregiver via the feedback device. Further, the settings associated with silence and happy sounds can be saved as pre-defined, preferred settings. In certain embodiments, the swing device 620 can be configured to automatically adjust its settings to preferred settings in response to agitated sounds or crying. The monitoring device 602 can also be configured to continually modify the preferred, pre-defined settings as the child grows and his preferences change. Additional structural and programming details and variations of such embodiments of the monitoring/feedback system 600 are described herein with respect to FIGS. 11-15.

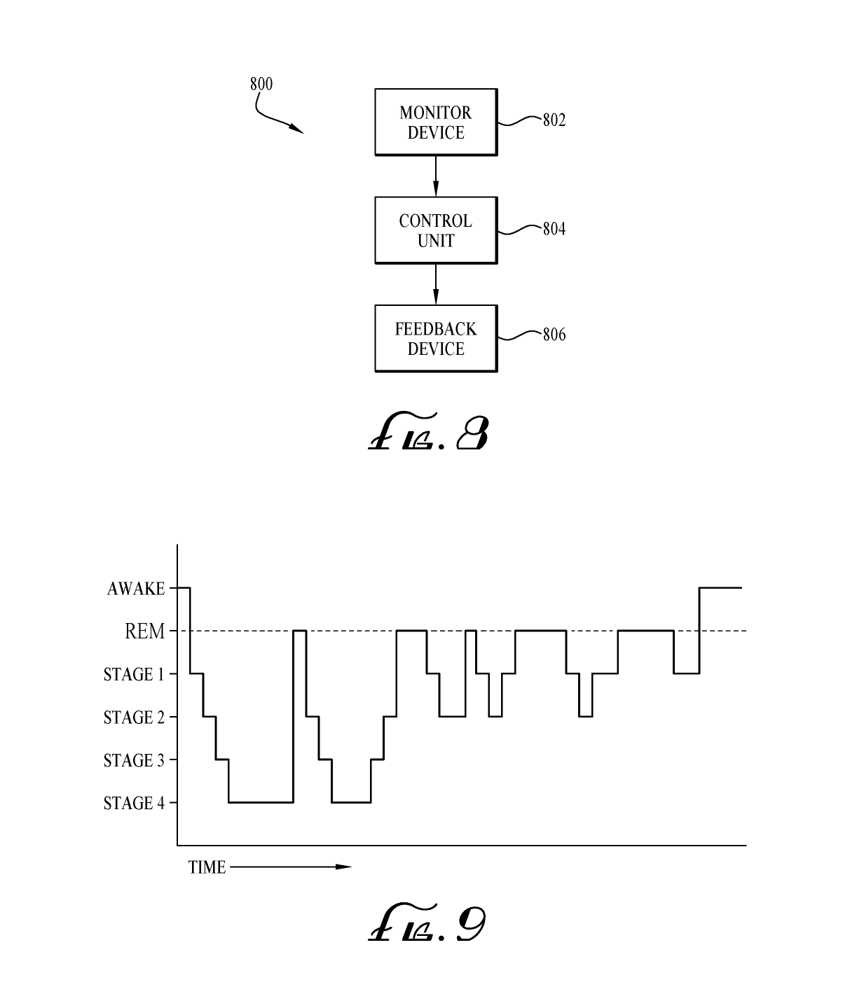

FIG. 7 shows a monitoring/feedback system 700 according to a seventh example embodiment. The monitoring/feedback system 700 of this embodiment includes an exercise monitoring device 702 coupled to or incorporated into a children's entertainer device 726. The depicted entertainer 726 includes a seat 728 suspended over a jump pad 730. The feet of a child who is positioned in the seat 728 hang down below the seat and engage the jump pad 730. Example embodiments of the exercise monitoring device 702 can be connected with a jiggle switch 732 configured to detect movement of the jump pad 730, thereby providing an indicator of the child's physical activity (e.g., the motion data can be saved on a storage device and/or analyzed/interpreted by a programmed processor to provide total active minutes per day, etc.). The exercise monitoring device 702 can relay to a user, for example, that the child was active for 30 minutes that day. In another embodiment, the exercise monitoring device 702 can additionally or alternatively include a pressure sensor 734 configured to detect the amount of pressure on the jump pad 730, an indicator of the child's leg strength (e.g., when the child in the seat is jumping up and down with a foot positioned on the jump pad, a programmed processor can analyze/interpret the input pressure data). The device 702 can relay to a user, for example, that the child's leg strength has increased by 25% over the last month. The exercise monitoring device 702 can also be configured to compare (e.g., using a programmed processor) the measured data to pre-set, exemplary data (e.g., stored and accessible in a local or remote database). Thus, the exercise monitoring device 702 can relay to a user, for example, that the child's leg strength is above average for children in the same age range. The fitness or other measured data by the input devices/sensors of the exercise monitoring device 702 can be relayed to and output by a feedback device (not shown; e.g., the remote feedback device 304 of FIG. 3), which can be attached to or separate from the exercise monitoring device 702. Additional structural and programming details and variations of such embodiments of the monitoring/feedback system 700 are described herein with respect to FIGS. 11-15.

Referring back to FIG. 2, multiple monitoring devices 202 and 206 can be adapted to relay information to a single, consolidated feedback device 204. For example, the sleep monitoring device 402, learning monitoring device 502, swing monitoring device 602, and/or exercise monitoring device 702 of respective FIG. 4, 5, 6, or 7 can be configured to relay information to the feedback device 304 of FIG. 3. As described above, the feedback device 304 can communicate the information to a caregiver, pediatrician, or expert. The feedback device 304 can also include pre-programmed milestones (e.g., counting to 10) and can determine when a child achieves the milestones and alert a caregiver. Further, a caregiver can select other people to share achieved milestones with. In example embodiments, a caregiver can create a contact list (e.g., email addresses, phone/text numbers, etc.) of those persons who wish to receive milestone achievement notifications, and the processor is programmed to send communications automatically (without user intervention) to those contacts upon determining that a milestone has been achieved. For example, a caregiver can add grandparents, aunts, uncles, and friends to the email list so that they are automatically informed about the child's ongoing accomplishments.

FIG. 8 shows a child sleep monitoring/feedback system 800 according to an eighth example embodiment. The monitoring/feedback system 800 includes a monitoring device 802, a control unit 804, and a feedback device 806. The monitoring device 802 is configured for sensing/inputting data related to a child's sleep cycle and communicating it to the control unit 804, which receives the monitored data from the monitoring device, processes and interprets it, and based thereon communicates with and controls the feedback device 806. The output device 806 includes one or more conventional output devices such as an alarm. The sleep monitoring/feedback system 800 can additionally or alternatively be configured to monitor other factors related to the child's sleep, such as, for example, sleep quality, duration, and/or patterns.

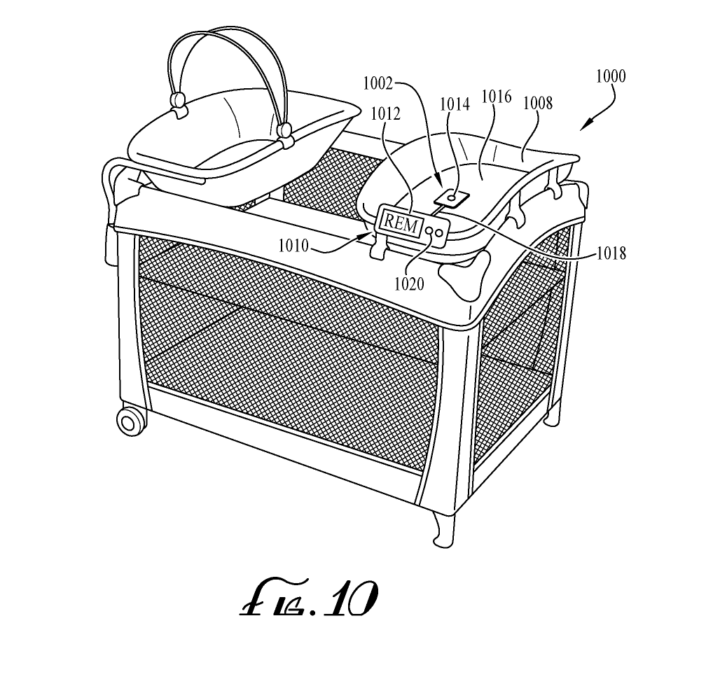

Before describing addition details of example embodiments, some background sleep information will be provided. A traditional sleep cycle has five stages: Stages 1, 2, 3, 4, and REM (rapid eye movement) sleep. Typically, the sleep cycle starts with Stage 1, progresses through stages 2, 3, and 4 until REM sleep is achieved, and then starts again at Stage 1. However, it is not uncommon for stages to be skipped or experienced in an alternative sequence. As shown in FIG. 9, for example, after completing the first sleep cycle in normal sequence, an individual may transition from REM sleep to Stage 2, skipping Stage 1. A complete cycle typically takes between about 90 and 110 minutes for an adult and between about 50 and 60 minutes for an infant. Further, adults typically spend approximately 20% of their sleep time in REM sleep, while infant spent approximately 50% of their sleep time in REM sleep. Stages 1 and 2 are periods of light sleep, Stages 3 and 4 are periods of deep sleep, and REM is a period of “paradoxical” sleep, wherein brain activity increases while muscles become more relaxed. It is difficult and undesirable to be awoken during deep sleep or paradoxical sleep; as such, the ideal wake time is during a light sleep stage.

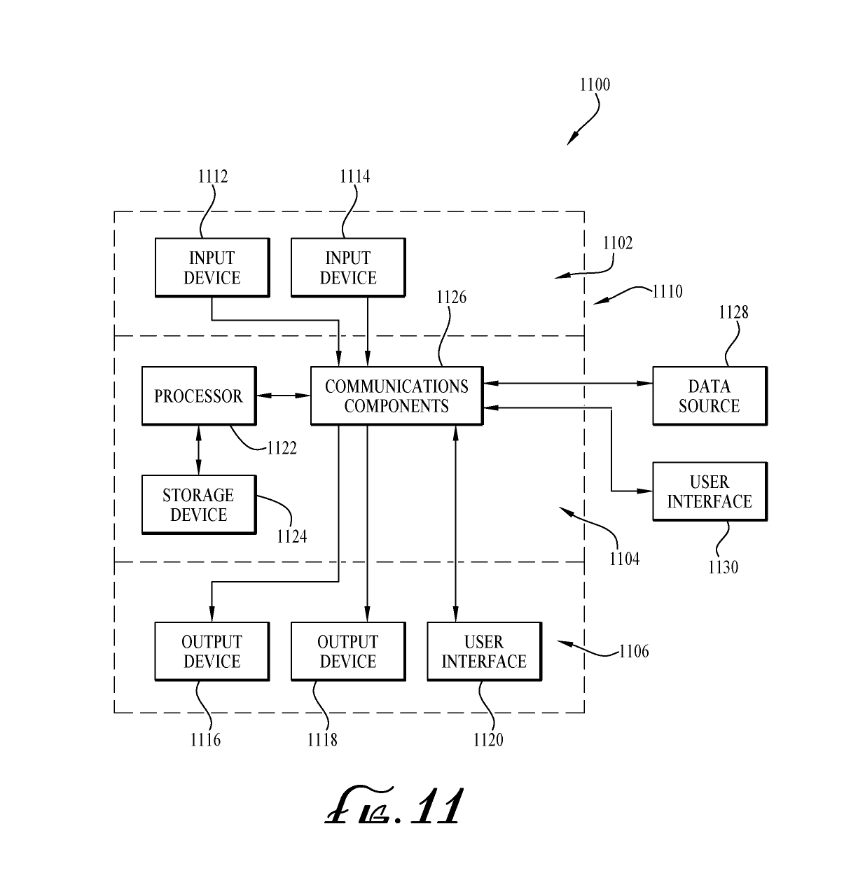

FIG. 10 shows a child sleep monitoring/feedback system 1000 according to a ninth example embodiment. The monitoring/feedback system 1000 includes a sleep monitoring device 1002 and a control hub 1010 coupled to a conventional child's sleep unit 1008. The control hub 1010 includes a control unit and a feedback device. The monitoring device 1002 is configured for sensing/inputting data related to a child's sleep cycle and communicating it to the control unit, which receives the monitored data from the monitoring device, processes and interprets it, and based thereon communicates with and controls the feedback device.

The feedback device includes one or more conventional output devices (not shown) for affecting the child in the sleep unit, such as an audio unit (e.g., a speaker or sound machine), an alarm unit (e.g., a speaker and/or light), a light, and/or an aromatherapy device. The feedback device can also include one or more conventional user interface devices for providing information to and/or receiving it from a caregiver, such as an alarm unit (e.g., a speaker) and/or a visual display screen 1012 for displaying the status of a child's sleep cycle, etc. The monitor 1002 can include one or more sensors 1014 configured to track signals indicative of the sleep stage that a child occupant is experiencing. For example, the sensor 1014 can include a motion sensor for monitoring the motions of a sleeping child occupant. Alternatively or additionally, the motion sensor 1014 can monitor the motions of a sleep surface 1016 on which the child is disposed. Motion is an indicator related to sleep cycles, as the movements of an individual vary with each different stage of sleep. In different embodiments, the sensor 1014 can be selected and configured to monitor other indicators related to sleep cycles, such as heart rate, breathing rate, brain activity, body temperature, etc. The monitor 1002 can be coupled to or incorporated into, for example, a child support device (such as the sleep unit 1008), a wearable device (such as a bracelet), or bedding (such as a sheets or a blanket). Other examples of child support devices which could accommodate such a monitor 1002 are play yards, cribs, bassinets, co-sleepers, inclined sleepers, and the like.

The control unit is in communication with and receives motion data from the monitor 1002. In the depicted embodiment, the control unit is housed within the control hub 1010. The motion data can be sent to the control unit via a wire transmitter 1018, as shown, or by another method, such as wireless transmission (in which case conventional wireless communication components are included such as a monitor transmitter/antenna and a control receiver/antenna using RF, BLUETOOTH, etc. wireless protocols). Thus, the control hub 1010 can be located proximate to the monitor 1002, as shown, or can be located away from the monitor, for example, remotely on a nightstand or in a caregiver's bedroom. The control unit includes a storage device and programmed processor configured to interpret the motion data to determine what stage of sleep the child is experiencing. In some embodiments, the control unit is also configured to count the number of sleep cycles completed.

In addition to or as an alternative to a control hub 1010, the monitor 1002 can be adapted to send the motion data (or other sleep-indicating data) to a smart device, for example, a tablet or a mobile phone. The smart device can include a software application for receiving and interpreting the sleep-indicating data then outputting (e.g., displaying) the sleep-stage data, or for merely displaying the sleep-stage data determined by the control unit. The software application can further be adapted to provide feedback to a caregiver on the child's sleep status, quality, etc. For example, the application can be adapted to alert a caregiver when the child wakes up in the middle of the night. In one embodiment, the application can alert the caregiver by sending a notification to the caregiver's mobile phone or by activating a sound alert. The application also can be adapted to control an output device, either automatically or based on user input, as described in further detail below. The output device (e.g., a soothing sound machine) can be integrated into the smart device, or can be separately contained. In some instances, the application is adapted for being downloaded onto the smart device by a caregiver.

As noted above, the feedback device includes one or more conventional output devices (not shown), housed within the control hub 1010, for affecting the child in the sleep unit. For example, in one embodiment, the output device is an alarm (e.g., a speaker and/or light) and the programmed processor is configured activate it to wake a child user up at a preset preferred time. Typically, the preferred time for waking a sleeping individual is when the individual shifts from REM sleep to a light sleep stage (i.e., Stage 1 or Stage 2). It is also preferred that a sleeping individual complete multiple sleep cycles per night. Thus, the preferred wake time can be preset to be, for example, during Stage 1 or 2 sleep and after a desired number of sleep cycles is completed. The alarm is coupled to the control unit such that, when the control unit determines that the user is in a light sleep stage and that a desired number of cycles have been completed (based on the sleep-indicating data received from the monitor 1002), the control unit activates the alarm.

As further noted above, the feedback device can also include one or more user interface devices that allow the child user or a caregiver user to selectively define components of the preferred wake time and/or to control other features of the monitoring system 1000. The user interface devices can include the visual display screen 1012 as well as pushbuttons 1020 for setting the parameters for the desired wake time for activating the alarm. For example, a user (child or caregiver) may prefer that the child be awoken during the first light sleep stage (stage 1 or 2) that occurs either after five sleep cycles or after 6 a.m., whichever occurs first. The user interface(s) and programmed processor can thus be configured to enable inputting these wake-up alarm parameters (or to enter an exact wake-up time as is conventionally done). In embodiments where the monitoring system 1000 also includes an audio unit (e.g., a speaker that converts into sounds the signals that it receives from the control unit), the user can select a preferred sleeping sound from a pre-set menu of soothing sounds saved on the storage device. For example, the pre-set menu of sounds may include white noise, running water, other nature sounds, piano music, other music, etc. Also, in addition to or in place of an alarm, the monitoring/feedback system 1000 can include a pre-set menu of waking sounds from which a user/caregiver can select a desired waking sound. The control unit can be adapted to turn off or slowly fade out the desired sleeping sound and turn on or slowly fade in the desired waking sound when the preferred wake time is reached. According to example embodiments, a user can download chosen sound options (e.g., a favorite song) to the audio unit for example from the connected mobile phone, tablet, or other device.

Moreover, example embodiments of the sleep monitoring/feedback system 1000 can include a noise-detection system for detecting ambient noise. The control unit can be adapted to automatically adjust the volume and/or frequency of the desired sleeping sound (e.g., produced by a speaker output device) in response to the ambient noise (e.g., detected by a microphone input device). For example, the volume and frequency of white noise can be automatically adjusted to effectively drown out music from an adjacent room or a police vehicle siren passing by. In another embodiment, the control unit can be adapted to automatically change between different sounds in response to ambient noise. For example, if the noise detection system detects that the child occupant is crying, the control unit can automatically change from white noise to the child's favorite lullaby, as pre-selected by a user.

Various alternative or additional output devices can be included in the sleep monitoring/feedback system 1000. In some embodiments, the control unit can be adapted to control a light output device, wherein the light is turned off or slowly lowered as a child user falls asleep and turned on or slowly raised at the preferred wake time. The control unit can further be adapted to control an aromatherapy output device, wherein soothing aromas are released during sleep and invigorating aromas are released at the preferred wake time. In still other embodiments, the various output devices can be manually turned on or off by a user, as desired. Additional structural and programming details and variations of such embodiments of the monitoring/feedback system 1000 are described herein with respect to FIGS. 11-15.

FIGS. 11-15 show a monitoring/feedback system 1100 according to a tenth example embodiment. It should be noted that any of the features of any of the previously described and shown embodiments can be included/implemented, individually or in any combination, by the monitoring/feedback system 1100. As such, this embodiment is enabled by the following disclosure in combination with the above disclosures, and the previously disclosed embodiments are enabled by their respective disclosures in combination with the following disclosure. In addition, it should be noted that while this and the other embodiments are described with respect to use for children, it will be understood they can be adapted for use by adults, particularly the sleep-related features.

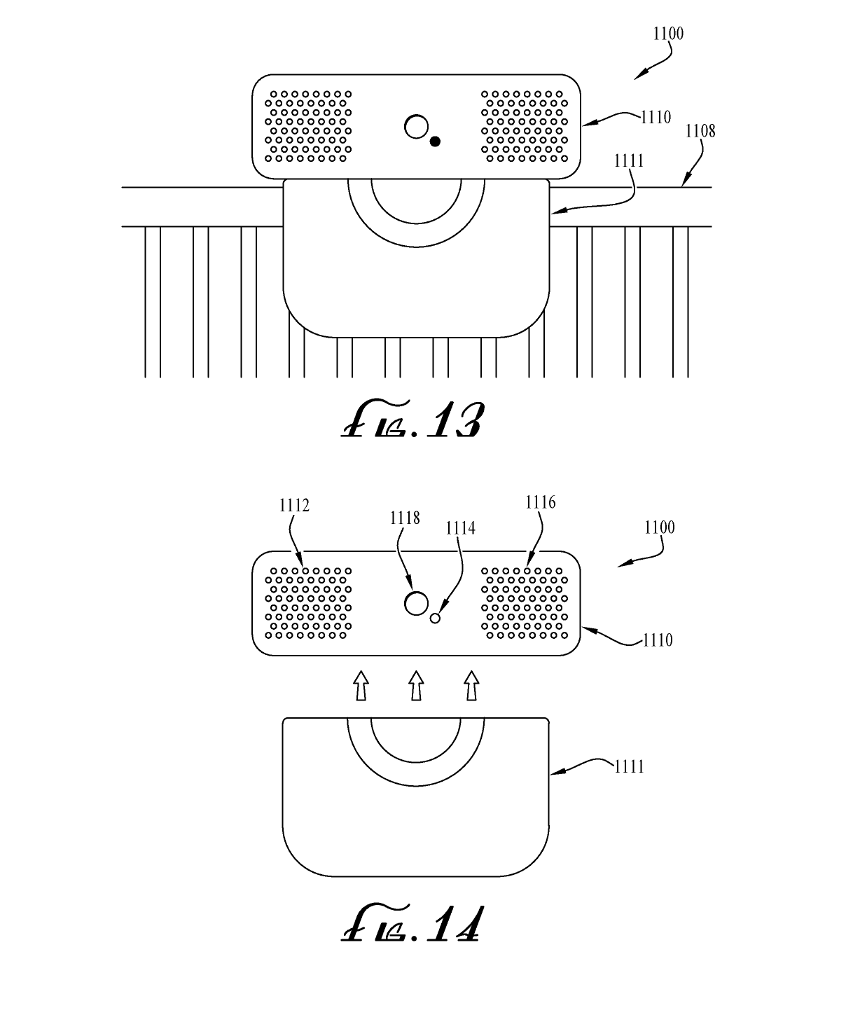

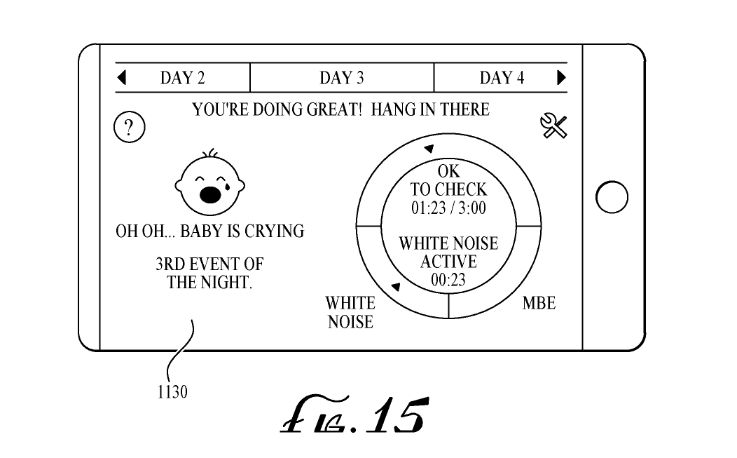

The monitoring/feedback system 1100 includes a sleep monitoring device 1102, a control unit 1104, and a feedback device 1106. In the depicted embodiment, these three components are integrated into a control hub 1110, which is detachably mounted to a mounting base 1111, which in turn is detachably mountable to a children's sleep unit 1108 (e.g., see FIGS. 13-14). The control hub 1110 and the mounting base 1111 can include conventional cooperating coupling components for detachably coupling together, for example a pin-and-well, hook-and-loop, tab-and-slop, deflectable finger and catch, or other arrangement known in the art. And the mounting base 1111 can include a conventional coupling component for detachably coupling to the sleep unit 1108, for example a spring-biased clamp or hook that can be placed onto a rail of a crib, bassinet, bouncer, or other child-holding device. In other embodiments, the monitoring device 1102, control unit 1104, and feedback device 1106 are each individual units not integrated together, or one of them can be separate and the other two integral (e.g., one of the input devices can be included in a separate bed pad, blanket, bracelet, etc.). And in other embodiments, the separate mounting base is not provided and the control hub includes an integral base for resting on a flat surface.

Referring particularly to FIG. 11, the monitoring device 1102 is configured for sensing/inputting data related to a child's sleep and communicating it to the control unit 1104, which receives the monitored data from the monitoring device, processes and interprets it, and based thereon communicates with and controls the feedback device 1106. As such, the monitoring device 1102 includes one or more input devices that receive inputs from the child occupant (e.g., measured data indicative of a physical condition of the child). The input devices can be provided by any of the monitoring input devices disclosed herein. In the depicted embodiment, the input devices include a microphone 1112 and a camera 114. The microphone 1112 and video camera 114 can be of a conventional type well known in the art, so for brevity of these components will not be described in detail. Additional or substitute input devices can be provided such as motion sensors, vibration sensors, electrodes (e.g., for detecting heart and/or breathing rate) moisture sensors (e.g., built into or attached to a diaper), and the like.

The monitoring device 1102 communicates with the control unit 1104. In typical embodiments, with these components integrated into the control hub 1110 or provided as separate components that are positioned near each other during use, the monitoring device 1102 communicates with the control unit 1104 by a wired connection (i.e., any electrical conductor). In other embodiments the monitoring device communicates with the control unit by a wireless connection, and as such the monitoring device includes wireless technology such as an antenna, a transmitter (or transceiver for two-way communicates such as to power on/off or adjust the input device), and a programmed processor of a conventional type.

Similarly, the feedback device 1106 includes one or more output devices that deliver outputs that affect the child occupant (e.g., in response to receiving control instruction sets from teh control unit to implement a sleep-training process). The output devices can be provided by any of the feedback output devices disclosed herein. In the depicted embodiment, the output devices include a speaker 1116 and a light 118. The speaker 1116 and light 118 can be of a conventional type well known in the art, so for brevity of these components will not be described in detail. In typical embodiments, the control unit 1104 can be set to automatically turn on the light 1118 when the camera 1114 is turned on. Additional or substitute output devices can be provided such as aroma-therapy devices, misters, humidifiers, vaporizers, vibrators (e.g., for transmitting vibrations through the sleep unit to the child), motion drives (e.g., to induce a rocking motion of a sleep unit with rockers), and the like.

The feedback device 1106 can optionally include one or more user interface devices 1120 such as output devices that deliver information (e.g., representing the sleep condition of the child) to the user (adult and/or child) and/or input devices that enable the user to set operational parameters of the monitoring/feedback system 1100. The interface devices can be provided by any of the interface devices disclosed herein, and they can be of a conventional type well known in the art, so for brevity of these components will not be described in detail. For example, the user interface devices 1120 can include a touch screen (for input and output) and a speaker. Other interfaces can include push buttons, rotary or slide knobs, microphones, lights, and the like. The interfaces 1120 are configured to provide for on/off control of the control unit, to output operational information such as the sleep stage of the child occupant, the output(s) currently on, and/or other sleep-related data, and to enter preset operating parameters such as output sequences (e.g., as described below) and wake-up parameters (e.g., as described above).

The control unit 1104 communicates with the feedback device 1106. In typical embodiments, with these components integrated into the control hub 1110 or provided as separate components that are positioned near each other during use, the control unit 1104 communicates with the feedback device 1106 by a wired connection (i.e., any electrical conductor). In other embodiments the control unit communicates with the feedback device by a wireless connection, and as such the feedback device includes wireless technology such as an antenna, a transmitter (or transceiver), and a programmed processor of a conventional type.

Further similarly, the control unit 1104 includes a processor 1122, a storage device 1124, and communication components 1126. These elements can be of a conventional type commonly used in consumer and children's products, so for brevity they will not be detailed. The storage device 1124 includes non-transitory memory that stores control software with instruction sets that are readable by the processor for implementing the functionally described herein (including to analyze the measured physical-condition data to determine a sleep condition of the child, and based on that control the output devices to run a sleep-training process). The memory optionally stores data such as preset (default or user selected) output sequences, preset wake-up parameters, user information (e.g., the name and birth-date/age of the child), and other data for implementing the functionality described herein. The communication components 1126 can be provided by a wired connection (i.e., any electrical conductor) in embodiments in which all components that interact are local to each other. In other embodiments, the control unit communicates with one or more of the monitoring device 102, the feedback device 106, a remote data source 1128, and a remote user interface device 1130 by a wireless connection, and as such the communication components 1126 include wireless technology such as an antenna and a transceiver that are controlled by the programmed processor.

The remote data source 1128 can be a database, website, or other data source on a remote computer that is connected for communications with the control unit 1104. For example, the remote data source 1128 can be Internet-accessed by the control unit 1104 to look up the prescribed amount of REM sleep for a five-year-old child, or the length of time parents should allow a newborn to cry before tending to it, based on the latest research, then input that updated data into the local storage device 1124 for use in default output sequences.

And the remote user interface device 1130 can be a feedback device (such as that of FIG. 3) that can be located in the caregiver's bedroom to remotely control the control unit 1104. Additionally or alternatively, the remote user interface device 1130 can be a mobile software application loaded onto a user's electronic device (e.g., smart phone, tablet). For example, the communications components 1126 of the control unit 1104 can use BLUETOOTH technology to communicate with the smart phone. The mobile software application can be optimized for use on mobile devices and include output screen displays and input touch buttons, for example as shown in FIG. 15.

The mobile or control software application can provide for customizing preloaded programs (e.g., entering preset output sequences from a menu of options), entering custom programs, and/or overriding current program actions and response. The storage device can include one or more pre-installed sleep-training programs/protocols (e.g., preset output sequences) for beginning use, and the mobile or control software application can provide for the user inputting history, habits, and goals, and offering an initial sleep-training program based on those inputs. The mobile or control software application can include learning and adapting features, for example the behavior of the child and the caregiver can be monitored and assessed over time then used to refine and course correct the running sleep-training program. Or the system can be run in a more passive timer mode. And the mobile or control software application can provide for support services such as community forums, training videos, articles and research, and/or coaching chat sessions.

In addition, the system 1100 can be in communication with and used to manually or automatically control other home convenience technologies such as HVAC systems, TVs, etc. The mobile or control software can be programmed to send alerts to the user interfaces 1120 and/or 1130 based on preset parameters, for example sleep events such as the child occupant crying.

Referring particularly to FIG. 12, an operational sleep-training process 1200 of the monitor/feedback system 1100 will be described to illustrate the control software of the storage device 1124 for reading by the processor 1122. It will be understood that this is just one of multiple different sleep-training programs that can be implemented by the monitor/feedback system 1100, and this example is representative and provided for illustration purposes only.

When the monitor/feedback system 1100 is powered on and a sleep-training program (e.g., preset output sequences) selected, at 1202 one or more of the input devices (e.g., the microphone 1112 and/or the video camera 1114) receive inputs from the child such as detected sounds and motions (e.g., measured data indicative of a physical condition of the child, such as images, sounds, motion, and temperature). Then at 1204 the control processor 1122 analyzes the received inputs using algorithms (e.g., of a conventional and well-known type) on the storage device 1124 to evaluate and determine the child occupant's sleep condition (e.g., crying, restless, quietly awake, REM or stage 1 sleep). If it is determined that the child is sleeping well at 1206, then no action is needed, and the process continues with further inputs being received until a sleep event is detected or until the sleep period is concluded.

If however it is determined that the child is sleeping poorly at 1206, then at 1208 a first output device (e.g., the speaker 1116) is activated to play a sound(s) (e.g., white noise, nature sounds, or a lullaby) to soothe and calm the child. The sound can be user-selected from a menu of sounds preloaded in the storage device 1124 or uploaded by the user. If at 1210 it is determined that the child is now sleeping well (based on fresh inputs being analyzed as just described), then at 1212 the data is saved to the storage device 1124 to record the sleep event and the effective output action taken. Next at 1214 the sleep-training program can optionally be updated to ensure that the effective output action taken is prioritized/high in the sequence of outputs. The sleep-training process 1200 then continues, receiving and analyzing further inputs to monitor the child's sleep condition.

If however it is determined that the child is still sleeping poorly at 1210, then at 1216 the sleep-training program is checked to determine if there are any more preset outputs in the sequence. If there are, then at 1218 the next output is activated to try soothing and calming the child. The next output can be the same device but with adjusted outputs (e.g., the speaker volume or song can be adjusted) or it can be a different device (e.g., the light 1118). The next output can be user-selected from a preinstalled menu or uploaded to the menu by the user. The process then returns to 1210 to continue trying to find an output action that has the desired soothing effect on the child.

If at 1216 it is determined that there are no more preset outputs in the sequence, then an alert is sent to the user interface 1120 or 1130. This can be the case where a prescribed sequence has run its full course without finding an effective output action, or where the user has preset only one or a few output actions. The alert can be a sound and/or display sent to the user interface 1120 or 1130, and/or a text or email to the remote interface 1130. The caregiver can then go into the child's room and tend to the child to calm and sooth the little one. The process can then be restarted back to the beginning, or it can continue running until a sleep event is detected or until the sleep period is concluded.

Other sleep-training programs can be implemented by the monitor-feedback system. In another similar example, the preset output sequence includes over time automatically increasing the number of outputs, or providing increasingly longer delays after detecting crying, before sending an alert to the caregiver. Current teaching is for parents to wait longer and longer periods after hearing crying to tend to the child to train them to calm themselves and not rely on the parents immediately coming to their rescue. The remote data source 1128 can be used to update the sleep-training program as teachings evolve over time. To implement this version of the sleep-training process 1200 of FIG. 12, at 1208 the first preset output run after detecting crying (or another undesired sleep condition of the child) is activated after a delay that is longer than the delay for any previously run output, using a clock feature of the monitoring/feedback system 1100, until the crying stops (or another desired sleep condition is detected) or until there are no more presets in which case an alarm signal is activated to notify the caregiver. Additional details related to this “learning” type of sleep-training program are described above with respect to FIG. 4.

In another similar example, the control unit can be set to activate an output speaker, to provide a wake-up alarm, at a preset preferred time based on the child's sleep conditions. For example, the preset time can be when the individual shifts from REM sleep to a light sleep stage (i.e., Stage 1 or Stage 2). Additional details related to this sleep-training program are described above with respect to FIG. 10.

Various changes and modifications to such a children's monitoring/feedback system, beyond those explicitly mentioned herein, are contemplated as being within the scope of the present invention. Moreover, the particular configurations, materials of construction, and objectives described herein are merely exemplary and are in no way limiting. As such, while the invention has been described with reference to example embodiments, it will be understood by those skilled in the art that a variety of modifications, additions, and deletions are within the scope of the invention, as defined by the following claims.

What is claimed is:

1. A sleep-training system for a child, comprising:
at least one input device configured to measure data indicative of a physical condition of the child;
a control unit including a storage device and a programmed processor configured to analyze the measured physical-condition data, determine a sleep condition of the child, and based thereon run a sleep-training process including a control instruction set; and
at least one output device configured to receive from the control unit the control instruction set and in response be operated and controlled to produce outputs affecting the child according to the sleep-training process.

2. The system of claim 1, further comprising at least one user interface configured to receive from the control unit data representing the sleep condition of the child and output the sleep-condition data to a caregiver.

3. The system of claim 2, wherein the at one user interface includes a local user interface integrated with the control unit, a remote user interface with communication components for communicating with communication components of the control unit, a software application for loading onto a mobile electronic device, or a combination thereof.

4. The system of claim 1, wherein the at least one input device is selected from the group consisting of a microphone, a camera, a motion sensor, a low-power radar, a thermometer, and an electrode.

5. The system of claim 1, wherein the measured physical-condition data is selected from the group consisting of sound, images, motion, temperature, heart rate, breathing rate, and brain activity of the child, and wherein the determined sleep condition of the child is selected from the group consisting of crying, restless, quietly awake, and asleep including sleep stages.

6. The system of claim 1, wherein the at least one output device is selected from the group consisting of a speaker, a display screen, a vibrator, a light, and an aroma-therapy device.

7. The system of claim 8, wherein the at least one output device is a speaker, the storage device stores sounds for outputting by the speaker, and the sounds are selected from the group consisting of white noise, songs, and ambient-noise-canceling sounds.

8. The system of claim 1, wherein the determined sleep condition of the child includes sleep stages of sleep and the sleep-training process includes activating an output as a wake-up alarm based at least in part on the determined sleep stage condition of the child.

9. The system of claim 1, wherein the sleep-training process includes, upon determining the sleep condition of the child is undesired, activating the at least one output device with a longer delay than a previous delay in activating the at least one output device.

10. The system of claim 1, wherein the sleep-training process includes:
upon determining the sleep condition of the child is undesired, activating a first preset output;
upon determining the sleep condition of the child remains undesired, activating a next preset output; and
repeating the process until determining the sleep condition of the child is desired or the determining no more outputs are preset.

11. The system of claim 1, wherein the at least one input device further measures ambient noise, the at least one output is a speaker, and the control unit controls the speaker to output at least one sound that cancels the ambient noise.

12. A method of sleep-training a child, comprising:
measuring data indicative of a physical condition of the child by using at least one input device;
analyzing the measured physical-condition data to determine a sleep condition of the child, and based thereon run a sleep-training process including a control instruction set, by using a control unit including a storage device and a programmed processor; and
operating and controlling at least one output device according to the control instruction set to produce outputs affecting the child according to the sleep-training process.

13. The system of claim 12, wherein the at least one input device is selected from the group consisting of a microphone, a camera, a motion sensor, a low-power radar, a thermometer, and an electrode, wherein the measured physical-condition data is selected from the group consisting of sound, images, motion, temperature, heart rate, breathing rate, and brain activity of the child, wherein the determined sleep condition of the child is selected from the group consisting of crying, restless, quietly awake, and asleep including sleep stages, wherein the at least one output device is selected from the group consisting of a speaker, a display screen, a vibrator, a light, and an aroma-therapy device, and wherein if the at least one output device is a speaker then the storage device stores sounds for outputting by the speaker and the sounds are selected from the group consisting of white noise, songs, and ambient-noise-canceling sounds.

14. The system of claim 12, wherein the determined sleep condition of the child includes sleep stages of sleep and the sleep-training process includes activating an output as a wake-up alarm based at least in part on the determined sleep stage condition of the child.

15. The system of claim 12, wherein the sleep-training process includes, upon determining the sleep condition of the child is undesired, activating the at least one output device with a longer delay than a previous delay in activating the at least one output device.

16. The system of claim 12, wherein the sleep-training process includes:
upon determining the sleep condition of the child is undesired, activating a first preset output;
upon determining the sleep condition of the child remains undesired, activating a next preset output; and
repeating the process until determining the sleep condition of the child is desired or the determining no more outputs are preset.

17. The system of claim 12, wherein the at least one input device further measures ambient noise, the at least one output is a speaker, and the control unit controls the speaker to output at least one sound that cancels the ambient noise.

18. The system of claim 12, further comprising at least one user interface configured to receive from the control unit data representing the sleep condition of the child and output the sleep-condition data to a caregiver, and wherein the at one user interface includes a local user interface integrated with the control unit, a remote user interface with communication components for communicating with communication components of the control unit, a software application for loading onto a mobile electronic device, or a combination thereof.

19. A system for monitoring a child and providing feedback to a caregiver, the system comprising:
a monitoring device configured to measure information related to the cognitive or physical development of the child; and
a feedback device configured to receive from the monitoring device the measured information related to the cognitive or physical development of the child and output the measured information related to the cognitive or physical development of the child to the caregiver.

20. The system of claim 19, wherein the monitoring device includes at least one input device selected from the group consisting of a microphone, a camera, a motion sensor, a low-power radar, a thermometer, and an electrode, wherein the measured information the input device measures is selected from the group consisting of sound, images, motion, temperature, heart rate, breathing rate, and brain activity of the child, and wherein the feedback device includes at least one output device selected from the group consisting of a speaker, a display screen, a vibrator, and a light.
